(19) United States
(12) Patent Application Publication
(10) Pub. No.: US 2022/0347663 A1
(43) Pub. Date: Nov. 3, 2022

(54) FERROSILICATE SSZ-70 MOLCULAR SIEVE, ITS SYNTHESIS AND USE

(71) Applicant: CHEVRON U.S.A. INC., San Ramon, CA (US)

(72) Inventors: Christopher Michael LEW, Alameda, CA (US); Cong-Yan CHEN, Kensington, CA (US); Bi-Zeng ZHAN, Albany, CA (US); Stacey Ian ZONES, San Francisco, CA (US)

(21) Appl. No.: 17/241,204

(22) Filed: Apr. 27, 2021

(51) Int. Cl. B01J 29/88; C01B 33/26; B01J 23/44; C07C 5/27

(52) U.S. Cl. CPC B01J 29/88; C01B 33/2884; B01J 23/44; C07C 5/2775; C01P 2002/72; C07C 2529/88

(57) ABSTRACT

A ferrosilicate molecular sieve having the framework structure of SSZ-70 and a method of making the same is disclosed. The ferrosilicate molecular sieve can be used in processes for dewaxing paraffinic hydrocarbon feedstocks.

### FIELD

This disclosure relates to ferrosilicate molecular sieves of *-SVY framework type and methods for producing the same. In addition, this disclosure relates to a process for dewaxing a paraffinic hydrocarbon oil using a catalyst comprising the molecular sieve.

### BACKGROUND

Molecular sieves are classified by the Structure Commission of the International Zeolite Association according to the rules of the IUPAC Commission on Zeolite Nomenclature. According to this classification, framework-type zeolites and other crystalline microporous molecular sieves, for which a structure has been established, are assigned a three-letter code and are described, for example, in the “Atlas of Zeolite Framework Types” (Sixth Revised Edition, Elsevier, 2007).

SSZ-70 is one of the molecular sieves for which a structure has been established and materials of this framework type are designated as *-SVY. SSZ-70 is a polytype of MWW and can be viewed as a disordered ABC-type stacking of MWW-layers. The MWW framework structure is characterized by two independent multidimensional channel systems. One pore system is defined by two-dimensional 10-member ring (10-MR) sinusoidal channels. The other consists of 12-MR super-cages connected by 10-MR windows.

Presently, hydroisomerization catalysis typically involves a bi-functional catalyst with an acid function, and a precious metal (PM) function. Acidity is usually provided by a molecular sieve component and the PM function is very often provided by platinum or palladium deposited on and/or in the catalyst. Molecular sieves used in currently available catalysts are provided with specific content of aluminum and/or silica to control the acidity. These catalysts show very good activity, but also suffer from relatively high cracking.

The present disclosure is directed to isomorphously substituted (Fe for Al) *-SW molecular sieves, i.e., iron (Fe) in the tetrahedrally coordinated framework position instead of the typical aluminum (Al), and their use as hydroisomerization catalysts. Substitution of iron into the framework allows for the modification of molecular sieve acid properties and, as a result, provides a catalyst with superior properties when compared to the currently available catalysts. Performance advantages of the catalysts increased yield (lower cracking) and more favorable product distribution.

### SUMMARY

In a first aspect, there is provided a ferrosilicate molecular sieve of *-SVY framework type and, in its as-synthesized form, comprising an organic structure directing agent in its pores, wherein the organic structure directing agent is represented by Formula (1):

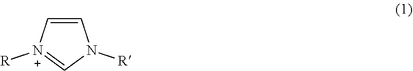

wherein R and R′ are each independently selected from isopropyl, isobutyl, and cyclohexyl.

In a second aspect there is provided a method of synthesizing a ferrosilicate molecular sieve of *-SVY framework type, the method comprising: (1) preparing a reaction mixture comprising: (a) a source of an oxide of iron; (b) a source of an oxide of silicon; (c) a source of an alkali or alkaline earth metal (M); (d) an organic structure directing agent (Q); (e) a source of hydroxide ions; and (f) water, and the reaction mixture has a composition, in terms of molar ratios, as follows:

| | |
|---|---|
| SiO₂/Fe₂O₃ | 25 to 750 |
| M/SiO₂ | 0.05 to 0.50 |
| Q/SiO₂ | 0.05 to 0.50 |
| OH/SiO₂ | 0.10 to 1.00 |
| H₂O/SiO₂ | 1 to 60 |

and (2) subjecting the reaction mixture to crystallization conditions sufficient to form crystals of the ferrosilicate molecular sieve; wherein the organic structure directing agent is represented by Formula (1):

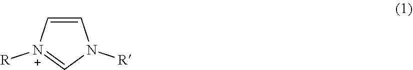

wherein R and R′ are each independently selected from isopropyl, isobutyl, and cyclohexyl.

In a third aspect, there is provided a process for hydroisomerization of a paraffinic hydrocarbon feedstock, the process comprising: contacting the paraffinic hydrocarbon feedstock at hydroisomerization conditions with hydrogen and a catalyst comprising a ferrosilicate molecular sieve of *-SVY framework type, and yielding a product having an increase in branched hydrocarbons relative to hydrocarbon feedstock; wherein the catalyst further comprises 0.01 to 10% by weight of a noble metal.

### BRIEF DESCRIPTION OF THE DRAWINGS

FIG. 1 shows a powder X-ray diffraction (XRD) pattern of as-synthesized ferrosilicate SSZ-70 prepared according to Example 1.

FIG. 2 shows a powder XRD pattern of calcined ferrosilicate SSZ-70 prepared according to Example 1.

FIG. 3 shows a powder XRD pattern of as-synthesized ferrosilicate SSZ-70 prepared according to Example 2.

FIG. 4 shows a powder XRD pattern of as-synthesized ferrosilicate SSZ-70 prepared according to Example 3.

FIG. 5(A) is a plot of conversion or yield versus temperature for the hydroconversion of n-decane over a Pd/Fe-SSZ-70 catalyst according to Example 22.

FIG. 5(B) is a plot of conversion or yield versus temperature for the hydroconversion of n-decane over a Pd/Al-SSZ-70 catalyst according to Example 22.

FIG. 6(A) is a plot of yield versus conversion for the hydroconversion of n-decane over a Pd/Fe-SSZ-70 catalyst according to Example 21.

FIG. 6(B) is a plot of yield versus conversion for the hydroconversion of n-decane over a Pd/Al-SSZ-70 catalyst according to Example 21.

FIG. 7(A) is a plot of C10 isomer distribution versus conversion for the hydroconversion of n-decane over a Pd/Fe-SSZ-70 catalyst according to Example 21.

FIG. 7(B) is a plot of C10 isomer distribution versus conversion for the hydroconversion of n-decane over a Pd/Al-SSZ-70 catalyst according to Example 21.

### DETAILED DESCRIPTION

### Definitions

The term “ferrosilicate” refers to a molecular sieve having a framework constructed of FeO4 and SiO4 tetrahedral units. The ferrosilicate may contain only the named oxides, in which case, it may be described as a “pure ferrosilicate” or it may contain other oxides as well.

The term “*-SVY” refers to an *-SVY topological type as recognized by the Structure Commission of the International Zeolite Association. Examples of *-SVY topological type materials include SSZ-70 and ECNU-5.

The term “as-synthesized” refers to a molecular sieve in its form after crystallization, prior to removal of the organic structure directing agent.

The term “Cn” hydrocarbon, wherein n is a positive integer (e.g., 1, 2, 3, 4, 5, etc.), means a hydrocarbon having n number of carbon atom(s) per molecule.

The term “Cn+” hydrocarbon, wherein n is a positive integer (e.g., 1, 2, 3, 4, 5, etc.), means a hydrocarbon having n or more than n carbon atom(s) per molecule.

The term “Cn—” hydrocarbon, wherein n is a positive integer (e.g., 1, 2, 3, 4, 5, etc.), means a hydrocarbon having no more than n carbon atom(s) per molecule.

The term “noble metal” as used herein generally refers to a metal selected from the group consisting of ruthenium, rhodium, palladium, silver, osmium, iridium, platinum, and gold.

The term “ferrosilicate SSZ-70” may be abbreviated as “Fe-SSZ-70”.

The term “aluminosilicate SSZ-70” may be abbreviated as “Al-SSZ-70”.

The term “M-SSZ-70” refers to a metallosilicate SSZ-70 having a framework constructed of SiO4 and MO4 tetrahedral units, where M is Al or Fe.

The term “1,3-diisobutylimidazolium” may be abbreviated as “DIBI”.

The term “1,3-dicyclohexylimidazolium” may be abbreviated as “DCHI”.

### Synthesis of the Molecular Sieve

A ferrosilicate molecular sieve of *-SVY framework type can be synthesized by: (1) preparing a reaction mixture comprising: (a) a source of an oxide of iron; (b) a source of an oxide of silicon; (c) an alkali or alkaline earth metal (M); (d) an organic structure directing agent (Q); (e) a source of hydroxide ions; and (f) water; and (2) subjecting the reaction mixture to crystallization conditions sufficient to form crystals of the ferrosilicate molecular sieve.

The reaction mixture can have a composition, in terms of molar ratios, within the ranges set forth in Table 1:

TABLE 1

| Reactants | Broadest | Secondary |
|---|---|---|
| SiO₂/Fe₂O₃ | 25 to 750 | 50 to 500 |
| M/SiO₂ | 0.05 to 0.50 | 0.10 to 0.50 |
| Q/SiO₂ | 0.05 to 0.50 | 0.10 to 0.50 |
| OH/SiO₂ | 0.10 to 1.00 | 0.10 to 0.50 |
| H₂O/SiO₂ | 1 to 60 | 5 to 40 |

Suitable sources of iron include water-soluble iron salts (e.g., ferric chloride, ferric nitrate, ferric sulfate).

Suitable sources of silicon include colloidal suspensions of silica, precipitated silica, fumed silica, alkali metal silicates, and tetraalkyl orthosilicates (e.g., tetraethyl orthosilicate).

The alkali or alkaline earth metal (M) is typically introduced into the reaction mixture in conjunction with the source of hydroxide ions. Examples of such metals include sodium and/or potassium, and also lithium, rubidium, cesium, magnesium, and calcium. As used herein, the phrase “alkali or alkaline earth metal” does not mean the alkali metals and alkaline earth metals are used in the alternative, but instead that one or more alkali metals can be used alone or in combination with one or more alkaline earth metals and that one or more alkaline earth metals can be used alone or in combination with one or more alkali metals.

The organic structure directing agent (Q) comprises a 1,3-dialkylimidazolium cation, represented by Formula (1):

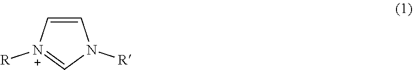

wherein R and R′ are each independently selected from isopropyl, isobutyl, and cyclohexyl. Specific examples of the organic structure directing agent include 1,3-diisopropylimidazolium cations, 1,3-diisobutylimidazolium cations, and 1,3-dicyclohexylimidazolium cations.

Suitable sources of Q include the hydroxides, chlorides, bromides, and/or other salts of the quaternary ammonium compound.

Optionally, the reaction mixture can contain a source of fluoride ions. The source of fluoride ions may be any compound capable of releasing fluoride ions in the reaction mixture. Examples of sources of fluoride ions include hydrogen fluoride; metal fluorides, preferably where the metal is sodium, potassium, calcium, magnesium, strontium or barium; ammonium fluoride; or tetraalkylammonium fluorides such as tetramethylammonium fluoride or tetraethylammonium fluoride. The molar ratio of F/SiO₂ in the reaction mixture can be in a range of from 0 to 1.0 (e.g., 0.01 to 1.0, 0.05 to 1.0, 0 to 0.5, 0.01 to 0.5, or 0.05 to 0.5).

The synthesis mixture may also contain seeds, typically of an *-SVY framework type molecular sieve, desirably in an amount from 0.01 to 10,000 ppm by weight (e.g., 100 to 5000 ppm by weight) of the reaction mixture. Seeding can be advantageous to improve selectivity for *-SW and/or to shorten the crystallization process.

Crystallization of the desired molecular sieve from the above reaction mixture can be carried out under either static, tumbled or stirred conditions in a suitable reactor vessel, such as for example polypropylene jars or Teflon-lined or stainless-steel autoclaves, at a temperature from 120° C. to 200° C. (e.g., 140° C. to 180° C.) for a time sufficient for crystallization to occur at the temperature used, e.g., from about 3 days to 30 days (e.g., 5 days to 25 days). Crystallization is usually conducted under pressure in an autoclave so that the reaction mixture is subject to autogenous pressure.

Once the desired molecular sieve crystals have formed, the solid product can be separated from the reaction mixture by standard mechanical separation techniques such as centrifugation or filtration. The recovered crystals are water-washed and then dried, for several seconds to a few minutes (e.g., 5 seconds to 10 minutes for flash drying) or several hours (e.g., 4 hours to 24 hours for oven drying at 75° C. to 150° C.), to obtain the as-synthesized molecular sieve crystals. The drying step can be performed under vacuum or at atmospheric pressure.

As a result of the crystallization process, the recovered crystalline molecular sieve product contains within its pores at least a portion of the structure directing agent used in the synthesis.

The as-synthesized ferrosilicate molecular sieve may be subjected to thermal treatment, ozone treatment, or other treatment to remove part or all of the organic structure directing agent used in its synthesis. Removal of the organic structure directing agent may be carried out using thermal treatment (e.g., calcination) in which the as-synthesized material is heated in air or inert gas at a temperature sufficient to remove part or all of the organic structure directing agent. While sub-atmospheric pressure may be used for the thermal treatment, atmospheric pressure is desired for reasons of convenience. The thermal treatment may be performed at a temperature at least 370° C. for at least a minute and generally not longer than 20 hours (e.g., from 1 to 12 hours). The thermal treatment can be performed at a temperature of up to 925° C. For example, the thermal treatment may be conducted at a temperature of 400° C. to 600° C. in air for approximately 1 to 8 hours.

The ferrosilicate molecular sieve (where part or all of the organic structure directing agent is removed) may be combined with a hydrogenating metal component. The hydrogenating metal component may be selected from molybdenum, tungsten, rhenium, nickel, cobalt, chromium, manganese, or a noble metal, such as platinum or palladium where a hydrogenation-dehydrogenation function is to be performed. Such hydrogenating metal components may be incorporated into the composition by way of one or more of the following processes: co-crystallizing; ion-exchanging into the composition; impregnating therein or physically admixing therewith. The amount of metal can be in a range of 0.001 to 20% (0.01 to 10%, or 0.5 to 2.0%) by weight of catalyst.

Once the ferrosilicate molecular sieve has been synthesized, it can be formulated into a catalyst composition by combination with another material resistant to the temperatures and other conditions employed in organic conversion processes. Such resistant materials may be selected from active materials, inactive materials, synthetic zeolites, naturally occurring zeolites, inorganic materials or a mixture thereof. Examples of such resistant materials may be selected from clays, silica, metal oxides such as alumina, or a mixture thereof. The inorganic material may be either naturally occurring, or in the form of gelatinous precipitates or gels, including mixtures of silica and metal oxides. Use of a resistant material in conjunction with the ferrosilicate molecular sieve, i.e., combined therewith or present during synthesis of the as-synthesized material, which crystal is active, tends to change the conversion and/or selectivity of the catalyst in certain organic conversion processes. Inactive resistant materials suitably serve as diluents to control the amount of conversion in a given process so that products can be obtained in an economic and orderly manner without employing other means for controlling the rate of reaction. These materials may be incorporated into naturally occurring clays (e.g., bentonite and kaolin) to improve the crush strength of the catalyst under commercial operating conditions. The inactive resistant materials (i.e., clays, oxides, etc.) function as binders for the catalyst. A catalyst having good crush strength can be beneficial because in commercial use it is desirable to prevent the catalyst from breaking down into powder-like materials.

Naturally occurring clays which may be composited with the ferrosilicate molecular sieve include the montmorillonite and kaolin family, which families include the subbentonites, and the kaolins commonly known as Dixie, McNamee, Georgia and Florida clays or others in which the main mineral constituent is halloysite, kaolinite, dickite, nacrite, or anauxite. Such clays may be used in the raw state as originally mined or initially subjected to calcination, acid treatment or chemical modification.

Binders useful for compositing with the ferrosilicate molecular sieve also include inorganic oxides selected from silica, zirconia, titania, magnesia, beryllia, alumina, or a mixture thereof.

In addition to the foregoing materials, the ferrosilicate molecular sieve may be composited with a porous matrix material such as silica-alumina, silica-magnesia, silica-zirconia, silica-thoria, silica-beryllia, silica-titania as well as ternary compositions such as silica-alumina-thoria, silica-alumina-zirconia, silica-alumina-magnesia and silica-magnesia-zirconia.

The relative proportions of ferrosilicate molecular sieve and inorganic oxide matrix may vary widely, with the molecular sieve content ranging from 1 to 95 percent by weight (e.g., 20 to 90 percent by weight) of the composite.

The catalyst is employed in the conventional manner in the form of, for example, spheres or extrudates.

### Characterization of the Molecular Sieve

In its as-synthesized and anhydrous form, the ferrosilicate molecular sieve can have a chemical composition, in terms of molar ratios, within the ranges set forth in Table 2:

TABLE 2

| | Broadest | Secondary |
|---|---|---|
| SiO₂/Fe₂O₃ | 25 to 750 | 50 to 500 |
| Q/SiO₂ | >0 to 0.1 | >0 to 0.1 |
| M/SiO₂ | >0 to 0.1 | >0 to 0.1 |

wherein Q comprises the organic structure directing agent described herein above and M is an alkali or alkaline earth metal.

As taught by U.S. Pat. No. 7,108,843, molecular sieve SSZ-70 has a powder X-ray diffraction pattern which, in its as-synthesized form, includes at least the peaks set forth in Table 3 below and which, in its calcined form, includes at least the peaks set forth in Table 4.

TABLE 3
Characteristic Peaks for As-Synthesized SSZ-70

| 2-Theta | d-spacing | Relative Intensity |
|---|---|---|
| [°] | [Å] | [100 × I/Io] |
| 3.32 | 26.6 | VS |
| 6.70 | 13.2 | VS |
| 7.26 | 12.2 | S |
| 8.78 | 10.1 | S |
| 13.34 | 6.64 | M |
| 20.02 | 4.44 | S |
| 22.54 | 3.94 | M |
| 22.88 | 3.89 | M |
| 26.36 | 3.38 | S-VS |
| 26.88 | 3.32 | M |

TABLE 4
Characteristic Peaks for Calcined SSZ-70

| 2-Theta | d-spacing | Relative Intensity |
|---|---|---|
| [°] | [Å] | [100 × I/Io] |
| 7.31 | 12.1 | VS |
| 7.75 | 11.4 | VS |
| 9.25 | 9.6 | VS |
| 14.56 | 6.08 | VS |
| 15.61 | 5.68 | S |
| 19.60 | 4.53 | S |
| 21.81 | 4.07 | M |
| 22.24 | 4.00 | M-S |
| 26.30 | 3.39 | VS |
| 26.81 | 3.33 | VS |

As will be understood by those skilled in the art, the determination of the parameter 2-theta is subject to both human and mechanical error, which in combination can impose an uncertainty of about ±0.15° on each reported value of 2-theta. The d-spacing values have a deviation determined based on the corresponding deviation ±0.15 degree 2-theta when converted to the corresponding values for d-spacing using Bragg's law. The relative intensities of the lines, l/lo, represents the ratio of the peak intensity to the intensity of the strongest line, above background. The relative intensities are given in terms of the symbols VS=very strong (>60), S=strong (≥40 and ≤60), M=medium (≥20 and <40), and W=weak (<20).

Minor variations in the diffraction pattern can result from variations in the mole ratios of the framework species of the particular sample due to changes in lattice constants. In addition, sufficiently small crystals will affect the shape and intensity of peaks, leading to significant peak broadening. Minor variations in the diffraction pattern can also result from variations in the organic compound used in the preparation. Calcination can also cause minor shifts in the XRD pattern. Notwithstanding these minor perturbations, the basic crystal lattice structure remains unchanged.

### Hydroisomerization of Paraffinic Hydrocarbon Feedstocks

The ferrosilicate molecular sieve is suitable for use as a catalyst in hydroisomerizing paraffinic hydrocarbon feedstocks when contacted by the catalyst with hydrogen at hydroisomerization conditions to yield a product having an increase in branched hydrocarbons relative to hydrocarbon feedstock.

Hydroisomerization conditions include a temperature from 200° C. to 450° C. (e.g., 250° C. to 400° C.), a pressure from 0.5 to 20 MPa (e.g., 1 to 15 MPa), a liquid hourly space velocity of from 0.1 to 10 h⁻¹ (e.g., 0.5 to 5 g⁻¹), and a hydrogen circulation rate of from 35.6 to 3560 Nm³/m³ (e.g., 356 to 1781 Nm³/m³).

The hydrocarbon feedstock is not limited to a specific type if the hydrocarbon feedstock includes n-C8+ hydrocarbons (e.g., n-C10+ hydrocarbons, or n-C15+ hydrocarbons). More specifically, examples of such hydrocarbon feedstocks include relatively light distilled fractions, such as kerosenes and jet fuels; and high boiling point stocks, such as fuel fractions or wax fractions derived from any type of crude oils, atmospheric distillation residues (atmospheric residues), vacuum tower residues, vacuum distillation residues (vacuum residues), cycle stocks, syncrudes (e.g., shale oil, tar oil, and the like), gas oil, vacuum gas oil, foots oil, and FT synthetic oil; and other heavy oils.

In some aspects, at least a portion of the feedstock can correspond to a feed derived from a biocomponent source. In this discussion, a biocomponent feed or feedstock refers to a hydrocarbon feedstock derived from a biological raw material component, such as vegetable fats/oils, animal fats/oils, fish oils, pyrolysis oils, and algae lipids/oils.

### EXAMPLES

The following illustrative examples are intended to be non-limiting.

### Example 1

### Synthesis of Ferrosilicate SSZ-70

A 23 mL Teflon liner was charged with 1.5628 g of deionized water, 0.1550 g of 50% aqueous NaOH, and 9.7013 g of a 1,3-dicyclohexylimidazolium hydroxide (10%). The mixture was stirred until a homogeneous solution was obtained. Then, 1.2 g of fumed silica was added slowly and stirred until a homogeneous solution was obtained. Finally, 0.1597 g of Fe(NO₃)₃.9H₂O was added. The resulting gel had a composition, in terms of molar ratios as follows:

1 SiO₂: 0.01 Fe₂O₃: 0.1 NaOH: 0.2 Q-OH :30 H₂O

The liner was capped and sealed with a 23 mL Parr autoclave vessel. The autoclave vessel was then heated in a convection oven under tumbling conditions for 7 days at 150° C. The product was isolated by filtration, washed with deionized water, and then dried in an oven at 95° C.

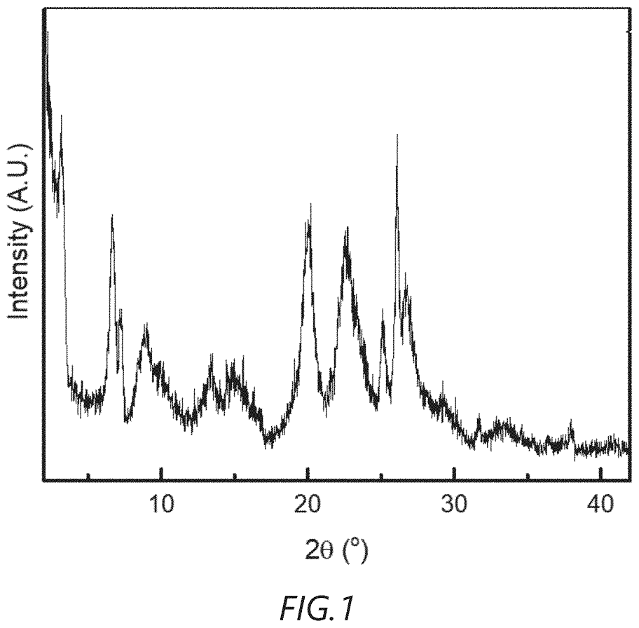

The product was pure SSZ-70 by powder XRD. FIG. 1 shows a powder XRD pattern of the as-synthesized product.

The as-synthesized product had a SiO₂/Fe₂O₃ molar ratio of 89, as determined by Inductively Coupled Plasma—Atomic Emission Spectroscopy Spectrometry (ICP-AES).

Thermogravimetric analysis (TGA) was performed by heating a sample under a flow of air and at a heating rate of 1° C./min between 20° C. and 900° C. TGA showed a mass loss of about 25%.

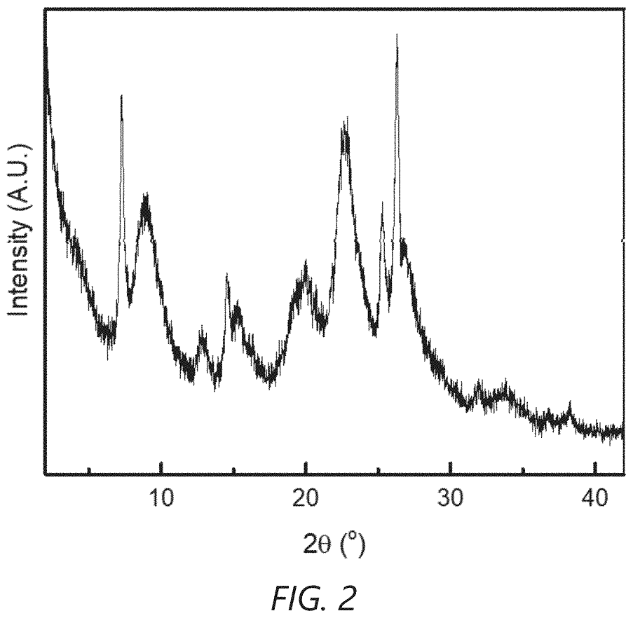

A sample of as-synthesized Fe-SSZ-70 was calcined in a muffle furnace by ramping the temperature to 120° C. at 1° C./minute in flowing air, holding the temperature at 120° C. for 2 hours, and then ramping the temperature to 550° C. in air at 1° C./minute. The temperature was held at 550° C. for 5 hours before each sample was allowed to cool to ambient temperature. FIG. 2 shows a powder XRD pattern of Fe-SSZ-70 in its calcined form.

A calcined sample (H-form) was measured with nitrogen physisorption and the data analyzed using the t-plot method. The material possessed a micropore volume of 0.17 cm³/g.

The isopropylamine temperature-programmed desorption (Ipam-TPD) was performed on the calcined material (H-form). Analysis of the data afforded an acid site density of 180 μma H⁺/g.

The physicochemical properties of the as-synthesized and calcined Fe-SSZ-70 materials are summarized Table 6 below.

### Example 2

### Synthesis of Ferrosilicate SSZ-70

A 23 mL Teflon liner was charged with 0.1808 g of 50% aqueous NaOH, and 9.9602 g of a 1,3-diisobutylimidazolium hydroxide (9%). The mixture was stirred until a homogeneous solution was obtained. Then, 1.4 g of fumed silica was added slowly and stirred until a homogeneous solution was obtained. Finally, 0.1854 g of Fe(NO₃)₃.9H₂O was added. A stream of nitrogen gas was blown over the mixture until the appropriate excess of water has been evaporated. The resulting gel had a composition, in terms of molar ratios as follows:

1 SiO₂: 0.01 Fe₂O₃: 0.1 NaOH: 0.2 Q-OH :20 H₂O

The liner was capped and sealed with a 23 mL Parr autoclave vessel. The autoclave vessel was then heated in a convection oven under tumbling conditions for 7 days at 150° C. The product was isolated by filtration, washed with deionized water, and then dried in an oven at 95° C.

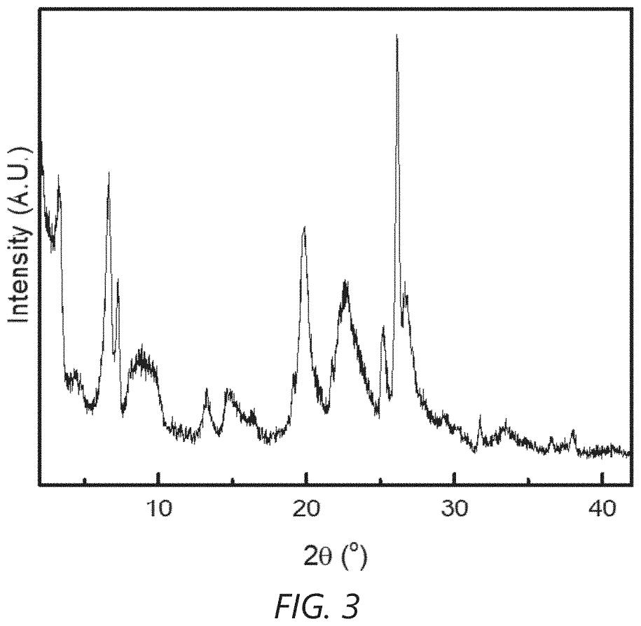

The product was pure SSZ-70 by powder XRD. FIG. 3 shows a powder XRD of the as-synthesized product.

### Example 3

### Synthesis of Ferrosilicate SSZ-70

A polyethylene bottle was charged with 5.0 g of tetraethylorthosilicate (TEOS) followed by 26.41 g of 1,3-diisobutylimidazolium hydroxide (9%). The mixture was allowed to stir overnight to hydrolyze the TEOS. A stream of nitrogen gas was blown over the mixture until the hydrolyzed ethanol had been evaporated. Then 0.4997 g of hydrofluoric acid (48%) and mixture homogenized with a spatula. Finally, 0.1977 g of Fe(NO₃)₃.9H₂O was added, and the mixture homogenize with a spatula. The resulting gel had a composition, in terms of molar ratios as follows:

1 SiO₂: 0.01 Fe₂O₃: 0.5 HF: 0.5 Q-OH :5 H₂O

The contents of the polyethylene bottle were transferred to a Teflon liner. The liner was capped and sealed with a 23 mL Parr autoclave vessel. The autoclave vessel was then heated in a convection oven under static conditions for 19 days at 150° C. The product was isolated by filtration, washed with deionized water, and then dried in an oven at 95° C.

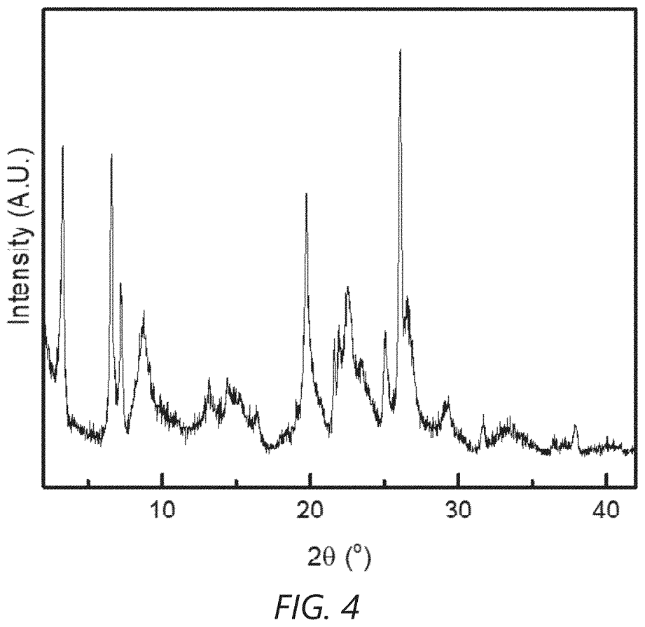

The product was pure SSZ-70 by XRD. FIG. 4 shows a powder XRD of the as-synthesized product.

### Examples 4-17

### Synthesis of Ferrosilicate SSZ-70

For preparing the Fe-SSZ-70 materials of Examples 4 to 17, the process conditions and molar ratios outlined in Table 5 below have been applied.

The respectively obtained materials were zeolitic materials having a framework structure of SSZ-70, as determined by powder XRD.

TABLE 5

| | Time | Temp. | | | SiO₂/ | Na/ | Q/ | OH/ | H₂O/ |
|---|---|---|---|---|---|---|---|---|---|
| Example | [days] | [° C.] | Rotation | Q | Fe₂O₃ | SiO₂ | SiO₂ | SiO₂ | SiO₂ |
| 4 | 22 | 150 | y | DIBI | 200 | 0.095 | 0.2 | 0.295 | 30 |
| 5 | 12 | 150 | y | DIBI | 500 | 0.092 | 0.2 | 0.292 | 30 |
| 6 | 7 | 150 | y | DCHI | 100 | 0.1 | 0.2 | 0.3 | 26 |
| 7 | 7 | 150 | y | DCHI | 100 | 0.1 | 0.2 | 0.3 | 30 |
| 8 | 7 | 150 | y | DCHI | 100 | 0.1 | 0.2 | 0.3 | 20 |
| 9 | 7 | 150 | y | DCHI | 100 | 0.1 | 0.2 | 0.3 | 40 |
| 10 | 7 | 150 | y | DCHI | 83 | 0.102 | 0.2 | 0.302 | 30 |
| 11 | 7 | 150 | y | DCHI | 67 | 0.105 | 0.2 | 0.305 | 30 |
| 12 | 7 | 150 | y | DCHI | 200 | 0.095 | 0.2 | 0.295 | 30 |
| 13 | 7 | 150 | y | DCHI | 500 | 0.092 | 0.2 | 0.292 | 30 |
| 14 | 7 | 150 | y | DCHI | 50 | 0.15 | 0.2 | 0.35 | 30 |
| 15 | 7 | 150 | y | DCHI | 50 | 0.2 | 0.2 | 0.4 | 30 |
| 16 | 7 | 150 | y | DCHI | 50 | 0.15 | 0.2 | 0.35 | 30 |

### Example 17

### Synthesis of Aluminosilicate SSZ-70

Aluminosilicate SSZ-70 (Al-SSZ-70) was prepared according to the procedure described by R. H. Archer et al. (Chem. Mater. 2010, 22, 2563-2572). A 23 mL Teflon liner was charged with 1.6071 g of water, 0.1550 g of NaOH (50%), and 9.7011 g of 1,3-dicyclohexylimidazolium hydroxide (10%). The mixture was stirred until homogeneous. Then, 38.4 mg of Reheis F-2000 aluminum hydroxide added to the mixture and stirred until homogeneous. 1.2 g of fumed silica was added to the mixture and stirred until homogeneous. The resulting gel had a composition, in terms of molar ratios, as follows:

1 SiO₂: 0.01 Al₂O₃: 0.1 NaOH: 0.2 Q-OH :30 H₂O

The liner was capped and sealed with a 23 mL Parr autoclave vessel. The autoclave vessel was then heated in a convection oven under tumbling conditions for 120 hours at 160° C. The product was isolated by filtration, washed with deionized water, and then dried in an oven at 95° C.

A sample of as-synthesized Al-SSZ-70 was calcined as described in Example 1.

The physicochemical properties of the as-synthesized and calcined Al-SSZ-70 materials are summarized Table 6 below.

TABLE 6
Physicochemical Properties of Fe-SSZ-70 and Al-SSZ-70

| | | Acid Site | Micropore |
|---|---|---|---|
| | SiO₂/M₂O₃ | Density | Volume |
| | Mole Ratio | [μmol/g] | [cm³/g] |
| Example 1 | | | |
| As-Synthesized Fe SSZ-70 | 89 | — | — |
| Calcined Fe-SSZ-70 | 91 | 180 | 0.17 |
| Example 17 | | | |
| As-Synthesized Al-SSZ-70 | 81 | — | — |
| Calcined Al-SSZ-70 | 91 | 270 | 0.17 |

### Example 18

### Ammonium Exchange

Calcined metallosilicate zeolites (M-SSZ-70), prepared according to Examples 1 and 17, were ion-exchanged to the ammonium form (NH₄⁺/M-SSZ-70) by adding the zeolite to a 10% NH₄NO₃ solution in a mass ratio of 10:1 10% NH₄NO₃ solution:M-SSZ-70 zeolite. The solution was heated at 95° C. for at least 2 hours. The solution was decanted and the process was repeated two more times. Following the final exchange, the zeolite was washed with deionized water to a conductivity of less than 50 μS/cm and dried. The resulting NR₄⁺/M-SSZ-70 can be converted to the hydrogen form (H⁺/M-SSZ-70) by calcination.

### Example 19

### Catalyst Preparation

Ammonium-exchanged metallosilicate SSZ-70 zeolites (NR₄⁺/M-SSZ-70) prepared according to Example 18 were ion-exchanged in an aqueous palladium nitrate solution at a pH of about 10 and at a Pd loading of 0.5 wt.%. The exchanged zeolite was washed with deionized water to a conductivity of less than 50 μS/cm and dried. The zeolite was then calcined in air at 482° C. for 3 hours.

### Example 20

### Constraint Index

The hydrogen-form metallosilicate SSZ-70 zeolites (H⁺/M-SSZ-70) was pelletized at 4-5 kpsi and crushed and meshed to 20-40. Then, 0.47 g of this catalyst (dry weight as determined by TGA at 600° C.) was packed into a ⅜ inch stainless steel tube with catalytically inactive alundum on both sides of the zeolite bed. An ATS (Applied Test Systems, Inc.) furnace was used to heat the reactor tube. Helium was introduced into the reactor at 23 mL/minute and atmospheric pressure. The catalyst was dehydrated at 482° C. for 2 hours. Then the reactor temperature was reduced to the pre-selected reaction temperature if necessary (e.g., 454° C.). The helium flow rate was then adjusted to 9.4 mL/minute and an equimolar mixture feed of n-hexane (n-C6) and 3-methylpentane (3-MP) was introduced into the reactor at a rate of 0.48 mL/h. The feed delivery was made via an ISCO pump. The on-line sampling of the product into a gas chromatograph (GC) began after 15 minutes of feed introduction. Representative results are shown in Table 7.

TABLE 7
Constraint Index Test Results

| | Example 1 | Example 17 |
|---|---|---|
| | [Fe-SSZ-70] | [Al-SSZ-70] |
| Temperature [° C.] | 482 | 454 |
| n-C6 Conversion [%] | 8.9 | 74 |
| 3-MP Conversion [%] | 8.4 | 73 |
| Total Feed Conversion [%] | 8.6 | 74 |
| Constraint Index | 1.1 | 1.0 |
| Iso-C4/n-C4 | 0.3 | 3.8 |

The results in Table 7 show that Al-SSZ-70 exhibits much higher conversions at a lower temperature than Fe-SSZ-70, which suggests that the acid sites associated with framework iron are weaker than the acid sites associated with framework aluminum. The Constraint Index values are essentially the same for both Al-SSZ-70 and Fe-SSZ-70, indicating that the differences in catalytic activity are not due to any differences in the framework.

### Example 21

### Hydroconversion of n-Decane

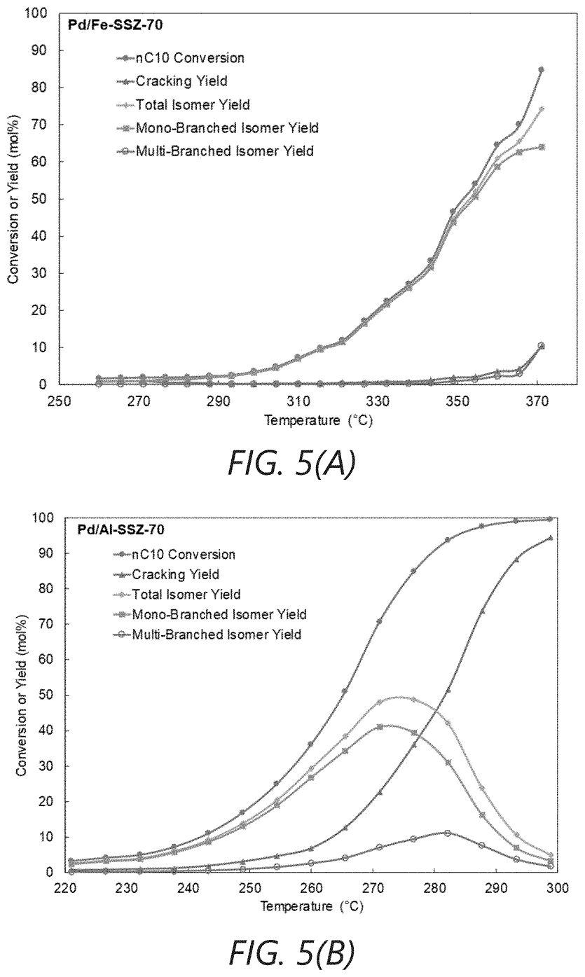

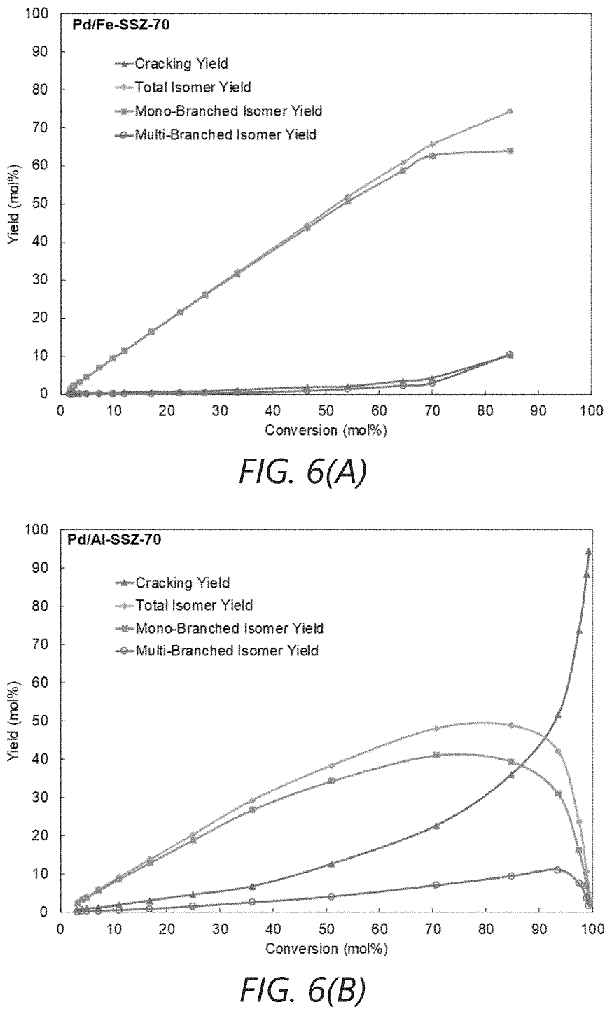

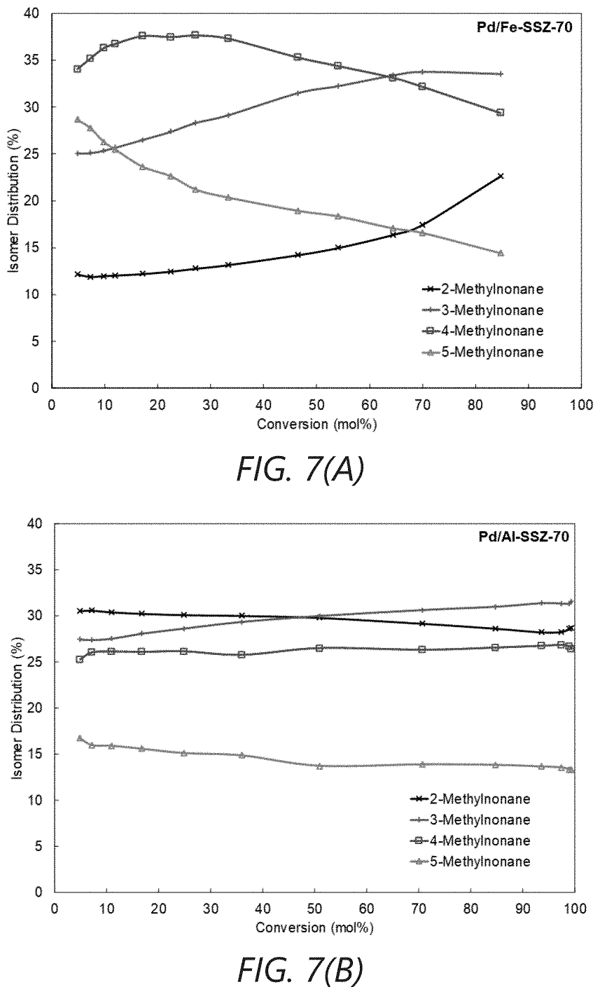

0.5 g of the Pd-loaded sample from Example 19 was pelletized at 5000 psi and meshed in a 20-40 range and charged into the center of a 23 inch-long ×¼ inch outside diameter stainless steel reactor tube with alundum loaded upstream of the catalyst for preheating the feed. The run conditions were as follows: a total pressure of 1200 psig; a down-flow hydrogen rate of 8.3 mL/minute, when measured at 1 atmospheric pressure and 25° C.; and a down-flow n-decane feed rate of 0.66 mL/h. All materials were first reduced in flowing hydrogen at about 315° C. for 1 h. Products were analyzed by on-line capillary gas chromatography (GC) once every thirty minutes. Raw data from the GC was collected by an automated data collection/processing system and hydrocarbon conversions were calculated from the raw data. Conversion is defined as the amount of n-decane reacted to produce other products (including iso-C10). Yields are expressed as mol% of products other than n-decane and include iso-C10 isomers as a yield product. The Fe-SSZ-70 n-decane hydroconversion results are compared to those of Al-SSZ-70 in FIGS. 5(A)-(B), 6(A)-(B) and 7(A)-(B).

1. A ferrosilicate molecular sieve of *-SVY framework type and, in its as-synthesized form, comprising an organic structure directing agent in its pores, wherein the organic structure directing agent is represented by Formula (1): 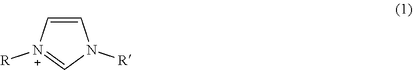
wherein R and R′ are each independently selected from isopropyl, isobutyl, and cyclohexyl.

2. The ferrosilicate molecular sieve of claim 1, having a molar ratio of SiO₂/Fe₂O₃ in a range of from 25 to 750.

3. The ferrosilicate molecular sieve of claim 1, having a molar ratio of SiO₂/Fe₂O₃ in a range of from 50 to 500.

4. A method of synthesizing a ferrosilicate molecular sieve of *-SVY framework type, the method comprising:
(1) preparing a reaction mixture comprising:
(a) a source of an oxide of iron;
(b) a source of an oxide of silicon;
(c) a source of an alkali or alkaline earth metal (M);
(d) an organic structure directing agent (Q);
(e) a source of hydroxide ions; and
(f) water, and the reaction mixture has a composition, in terms of molar ratios, as follows:

| | |
|---|---|
| SiO₂/Fe₂O₃ | 25 to 750 |
| M/SiO₂ | 0.05 to 0.50 |
| Q/SiO₂ | 0.05 to 0.50 |
| OH/SiO₂ | 0.10 to 1.00 |
| H₂O/SiO₂ | 1 to 60 |

and (2) subjecting the reaction mixture to crystallization conditions sufficient to form crystals of the ferrosilicate molecular sieve;
wherein the organic structure directing agent is represented by Formula (1): 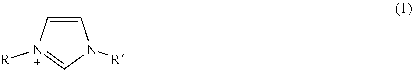
where R and R′ are each independently selected from isopropyl, isobutyl, and cyclohexyl.

5. The method of claim 4, wherein the reaction mixture has a composition, in terms of molar ratios, as follows:

| | |
|---|---|
| SiO₂/Fe₂O₃ | 50 to 500 |
| M/SiO₂ | 0.10 to 0.50 |
| Q/SiO₂ | 0.10 to 0.50 |
| OH/SiO₂ | 0.10 to 0.50 |
| H₂O/SiO₂ | 5 to 40. |

6. The method of claim 4, wherein the crystallization conditions include heating the reaction mixture under autogenous pressure at a temperature from 120° C. to 200° C. for 3 days to 30 days.

7. The method of claim 4, wherein the reaction mixture further comprises a source of fluoride ions.

8. The method of claim 7, wherein the reaction mixture has a molar ratio of F/SiO₂ in a range of from 0.01 to 1.0.

9. A process for hydroisomerization of a paraffinic hydrocarbon feedstock, the process comprising:
contacting the paraffinic hydrocarbon feedstock at hydroisomerization conditions with hydrogen and a catalyst comprising a ferrosilicate molecular sieve of *-SVY framework type, and yielding a product having an increase in branched hydrocarbons relative to hydrocarbon feedstock;
wherein the catalyst further comprises 0.01 to 10% by weight of a noble metal.

10. The process of claim 9, wherein the paraffinic hydrocarbon feedstock comprises n-C8+ hydrocarbons.

11. The process of claim 9, wherein the ferrosilicate molecular sieve has a molar ratio of SiO₂/Fe₂O₃ in a range of from 25 to 750.

12. The process of claim 9, wherein the ferrosilicate molecular sieve has a molar ratio of SiO₂/Fe₂O₃ in a range of from 50 to 500.

13. The process of claim 9, wherein the noble metal comprises platinum, palladium, or a mixture thereof.

14. The process of claim 9, wherein the hydroisomerization conditions include a temperature from 200° C. to 450° C., a pressure from 0.5 to 20 MPa, a liquid hourly space velocity of from 0.1 to 10 h⁻¹, and a hydrogen circulation rate of from 35.6 to 3560 Nm³/m³.
